(19) United States
(12) Patent Application Publication
(10) Pub. No.: US 2013/0280716 A1
(43) Pub. Date: Oct. 24, 2013

(54) Ratio of apoa2 to HDLc or Equivalents thereof, Risk Markers for Cardiovascular Disease

(71) Applicant: Stanley L. Hazen, Pepper Pike, OH (US)

(72) Inventor: Stanley L. Hazen, Pepper Pike, OH (US)

(73) Assignee: THE CLEVELAND CLINIC FOUNDATION, Cleveland, OH (US)

(21) Appl. No.: 13/884,577

(22) PCT Filed: Nov. 10, 2011
(86) PCT No.: PCT/US2011/060279

§ 371 Date: Jul. 10, 2013

Related U.S. Application Data
(60) Provisional application No. 61/412,387, filed on Nov. 10, 2010.

(51) Int. Cl. G01N 33/92

(52) U.S. Cl. CPC G01N 33/92
USPC 435/6.11; 436/501; 435/7.92

(57) ABSTRACT

The present disclosure provides methods and markers for characterizing a subject's, particularly a human subject's, risk of having cardiovascular disease. The present disclosure also provides methods of characterizing a subject's risk of developing cardiovascular disease. In another embodiment, the present disclosure provides methods for characterizing a subject's risk of experiencing a complication of cardiovascular disease or major adverse cardiac event within 3 months, 6 months, 1 year, 3 years, 5 years, or 10 years. In another embodiment, the present disclosure provides a method for determining whether a subject presenting with chest pain is at risk near term of experiencing a heart attack or other major adverse cardiac event. The present methods are especially useful for identifying those subjects who are in need of highly aggressive CVD therapies as well as those subjects who require no therapies targeted at inhibiting or preventing CVD or complications of CVD.

### STATEMENT REGARDING FEDERALLY SPONSORED RESEARCH

The work described in this disclosure was supported, at least in part, by Grant No. P01 HL76491 from the National Institutes of Health. The United States government has certain rights in these inventions.

### FIELD

The present disclosure relates to the field of cardiovascular disease. More specifically, it relates to methods and markers for determining whether a subject, particularly a human subject, is at risk of developing cardiovascular disease, having cardiovascular disease, or experiencing a complication of cardiovascular disease, e.g., a major adverse cardiac event. The present disclosure also relates to the use of such methods and markers for monitoring the status of cardiovascular disease in a subject or the effects of therapeutic agents on subjects with cardiovascular disease.

### BACKGROUND

Cardiovascular disease (CVD) is the general term for heart and blood vessel diseases, including, but not limited to, atherosclerosis, coronary heart disease, cerebrovascular disease, aorto-iliac disease, and peripheral vascular disease. Subjects with CVD may develop a number of complications or experience a major adverse cardiac event (MACE), including, but not limited to, the need for revascularization, myocardial infarction, stroke, angina pectoris, transient ischemic attacks, heart failure, aortic aneurysm, and death. CVD accounts for one in every two deaths in the United States. Thus, prevention of cardiovascular disease is an area of major public health importance.

A low-fat diet and exercise are recommended to prevent CVD. In addition, a number of therapeutic agents may be prescribed by medical professionals to those individuals who are known to be at risk for developing or having CVD. These include lipid-lowering agents that reduce blood levels of cholesterol and triglycerides, agents that normalize blood pressure, agents, such as aspirin or platelet ADP receptor antagonist (e.g., clopidogrel and ticlopidine), that prevent activation of platelets and decrease vascular inflammation, and pleiotropic agents such as peroxisome proliferator activated receptor (PPAR) agonists, with broad-ranging metabolic effects that reduce inflammation, promote insulin sensitization, improve vascular function, and correct lipid abnormalities. More aggressive therapy, such as administration of multiple medications or surgical intervention may be used in those individuals who are at high risk. Since CVD therapies may have adverse side effects, it is desirable to have methods for identifying those individuals who are at risk, particularly those individuals who are at high risk, of developing or having CVD.

Currently, several risk factors are used by medical professionals to assess an individual's risk of developing or having CVD and to identify individuals at high risk. Major risk factors for cardiovascular disease include age, hypertension, family history of premature CVD, smoking, high total cholesterol, low HDL cholesterol, obesity and diabetes. The major risk factors for CVD are additive, and are typically used together by physicians in a risk prediction algorithm to target those individuals who are most likely to benefit from treatment for CVD. These algorithms achieve a high sensitivity and specificity for predicting risk of CVD within 10 years. However, the ability of existing algorithms to predict a higher probability of developing CVD is limited. Among those individuals with none of the current risk factors, the 10-year risk for developing CVD is still about 2%. In addition, a large number of CVD complications occur in individuals with apparently low to moderate risk profiles, as determined using currently known risk factors. Thus, there is a need to expand the present cardiovascular risk algorithm to identify a larger spectrum of individuals at risk for or affected with CVD.

The mechanism of atherosclerosis is not well understood. Over the past decade a wealth of clinical, pathological, biochemical and genetic data support the notion that atherosclerosis is a chronic inflammatory disorder. Acute phase reactants (e.g., C-reactive protein, complement proteins), sensitive but non-specific markers of inflammation, are enriched in fatty streaks and later stages of atherosclerotic lesions. In a prospective clinical trial, base-line plasma levels of C-reactive protein independently predicted risk of first-time myocardial infarction and stroke in apparently healthy individuals. U.S. Pat. No. 6,040,147 describes methods which use C-reactive protein, cytokines, and cellular adhesion molecules to characterize an individual's risk of developing a cardiovascular disorder. Although useful, these markers may be found in the blood of individuals with inflammation due to causes other than CVD, and thus, these markers may not be specific enough. Moreover, modulation of their levels has not been shown to predict a decrease in the morbidity or mortality of CVD.

### BRIEF SUMMARY

The present disclosure provides methods and markers for characterizing a subject's, particularly a human subject's, risk of having or developing CVD. In another embodiment, the present disclosure provides methods for characterizing a subject's risk of experiencing a complication of CVD or a MACE within 1, 3, 5, and 10 years. The present methods and markers are also useful for identifying those subjects who are in need of highly aggressive CVD therapies, as well as those subjects who require no therapies targeted at inhibiting or preventing CVD or complications of CVD.

Apolipoprotein A2 (ApoA2), a high-density lipoprotein (HDL) associated protein, has been associated with triglyceride metabolism and insulin resistance in animal models. Despite being the second most abundant protein in HDL particles, its role in humans is unclear. Epidemiology studies have suggested a potential cardioprotective role for ApoA2, but without considering HDL as a confounding factor. The instant disclosure shows that apoA2, when adjusted for HDL, provides an independent association between apoA2 and having or developing CVD, rather than a cardioprotective role for apoA2.

In one embodiment, the present methods of determining the subject's risk of having or developing CVD comprise determining the ratio of apoA2 to HDL cholesterol (HDLc) or an equivalent thereof in a biological sample, e.g., a bodily fluid (for example, blood, serum, or plasma) obtained from a test subject, comparing the test subject's value to a control value that is derived from the ratio of apoA2 to HDLc or an equivalent thereof in comparable biological samples obtained from a control population, wherein test subjects whose ratio of apoA2 to HDLc or equivalent thereof is above the control value or in a higher range of control values are at greater risk of having or developing CVD than test subjects whose the apoA2/HDLc or an equivalent thereof is at or below the control value or in a lower range of control values.

In certain embodiments, the present risk marker is a ratio of apoA2/HDLc, which may be measured by the ratio of apoA2/HDL particle number (HDLp), apoA2/apolipoprotein A1 (apoA1), or apoA2/(apoA1+apoA2). In some embodiments, the present methods comprise determining the ratio of apoA2 mass/HDLc. In some embodiments, the apoA2 mass/HDLc ratio may be determined by the ratio of apoA2 mass/HDLp, apoA2 mass/apoA1, or apoA2 mass/(apoA1+apoA2) in a biological sample, for example, blood, serum, or plasma, from the subject. HDLp, apoA1, and (apoA1+apoA2) are considered equivalents of HDLc, and may be used interchangeably with HDLc to obtain the disclosed ratios of apoA2 to HDLc.

Also disclosed herein, are methods of determining a subject's risk of having or developing CVD comprising identifying at least one genetic determinant of apoA2 level in a test subject. In some embodiments, the at least one genetic determinant of apoA2 level is a single nucleotide polymorphism (SNP). In some embodiments, the SNP is selected from the group consisting of rs1262269 and rs70753770. If the test subject carries even one allele of an at risk SNP, which is associated with higher apoA2 levels, the test subject is at increased risk of having or developing CVD, and therefore at increased risk of experiencing CVD complications, a MACE, or both.

### BRIEF DESCRIPTION OF THE SEVERAL VIEWS OF THE DRAWINGS

FIG. 1 shows a distribution of ApoA2/HDL cholesterol.

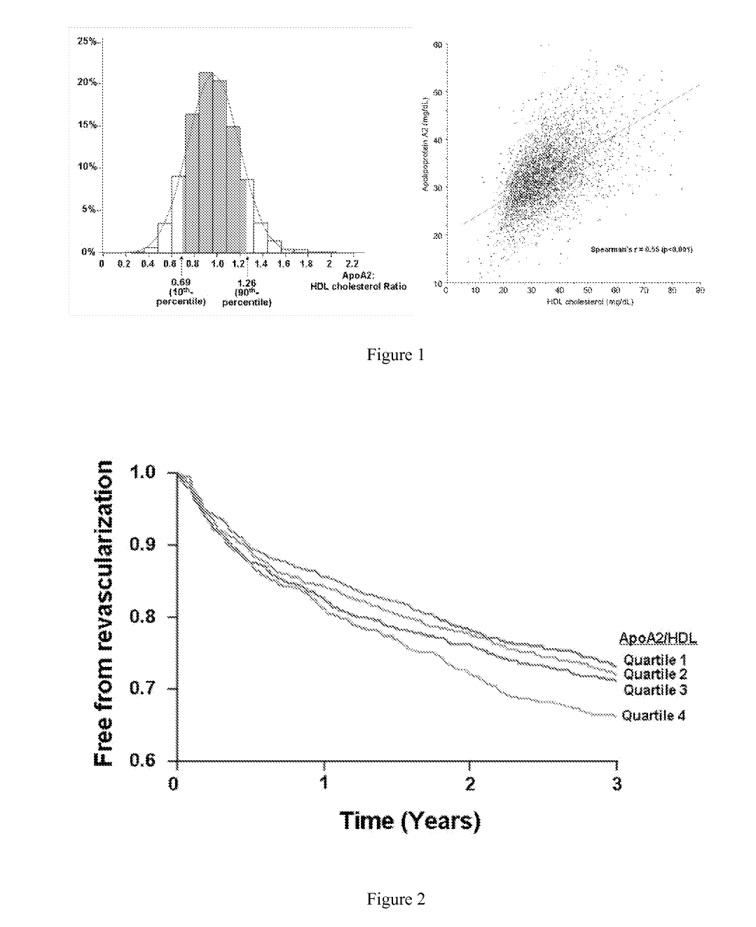

FIG. 2 shows free from revascularization according to ApoA2/HDL quartiles.

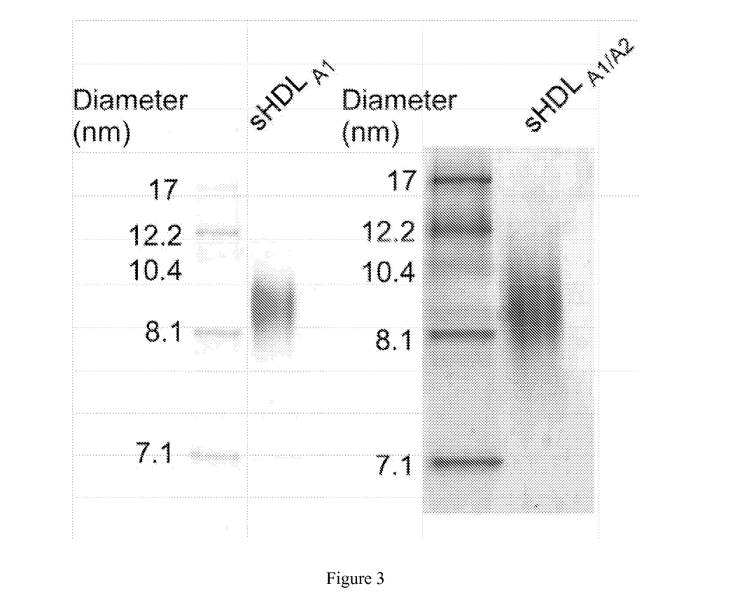

FIG. 3 shows characterization of reconstituted spherical HDL possessing only apoA1 (sHDLA1) versus both apoA1 and apoA1 (sHDLA1/A2). Isolated human apoA1 and apoA2 were used to generate reconstituted spherical HDL possessing either only apoA1 vs apoA1 and apoA2 according to the method of Kerry-Anne Rye and colleagues. Particle composition and protein content per particle were confirmed by biochemical assays and cross-linking mass spectrometry studies showing approximately 30:12:4:1 (mol:mol:mol:mol) phospholipid:CE:free cholesterol:apoA1 for sHDLA1 with 3 apoA1 per particle, and similar lipid/protein composition in A1/A2 sHDL except for 2 apoA1 and 2 apoA2 dimers per particle. Native gel PAGE analyses are shown, reveling similar sized (approx 9.6 nm) and relatively homogenous particles.

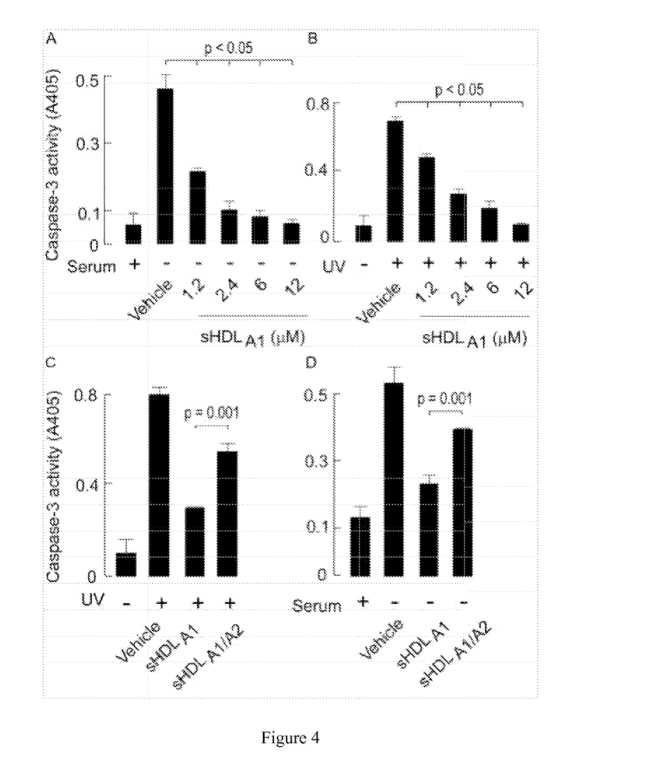

FIG. 4 shows Spherical HDL containing both apoA1 and apoA2 is less anti-apoptotic activity than spherical HDL containing only apoA1. A: HUVEC were placed in media in the presence vs absence of serum along with the indicated concentrations of spherical HDL containing only apoA1 and apoptosis was measured as a function of caspase-3 activation. B: HUVEC were exposed to 254-nm UV irradiation for 10 min followed by incubation with the indicated concentrations of spherical HDL containing only apoA1. Apoptosis was measured as a function of caspase-3 activation. C: HUVEC were exposed to 254-nm UV irradiation for 10 min followed by incubation with 6 μM spherical HDL containing apoA1 only or spherical HDL containing both apoA1 and apoA2. Apoptosis was measured as a function of caspase-3 activation. D: HUVEC were placed in serum-free medium along with 6 μM spherical HDL containing apoA1 only or spherical HDL containing both apoA1 and apoA2. Apoptosis was measured as a function of caspase-3 activation.

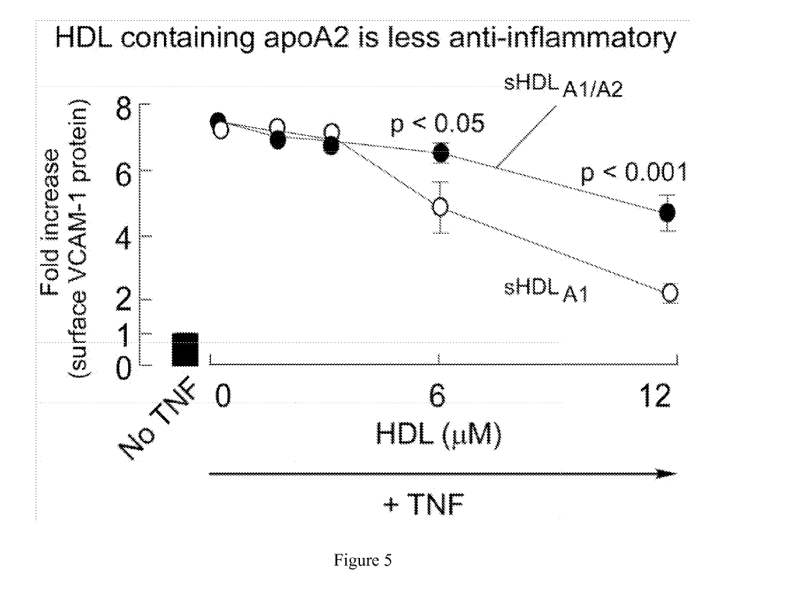

FIG. 5 shows spherical HDL containing both apoA1 and apoA2 is less efficient at inhibiting TNF-α induced surface VCAM-1 protein expression than spherical HDL containing only apoA1. HUVEC surface VCAM-1 protein expression was quantified by enzyme-linked immunosorbent assay in the presence and absence of TNF-α. In parallel, the impact of the indicated concentrations of spherical HDL containing only apoA1 or spherical HDL containing both apoA1 and apoA2 was determined by enzyme-linked immunosorbent assay.

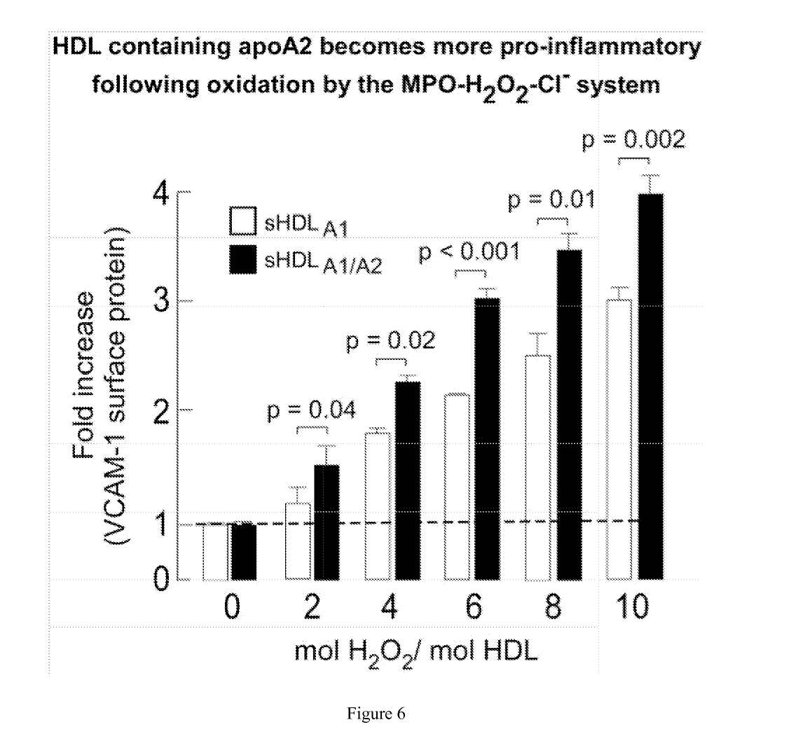

FIG. 6 shows spherical HDL containing both apoA1 and apoA2 is more pro-inflammatory than spherical HDL containing only apoA1. BAEC were incubated with apoA1 only containing spherical HDL or apoA1 and apoA2 containing spherical HDL that were previously exposed to the MPO-H2O2-Cl— system of activated leukocytes at the indicated mole ratios of hydrogen peroxide per HDL particle. Surface VCAM-1 protein expression was determined by enzyme-linked immunosorbent assay.

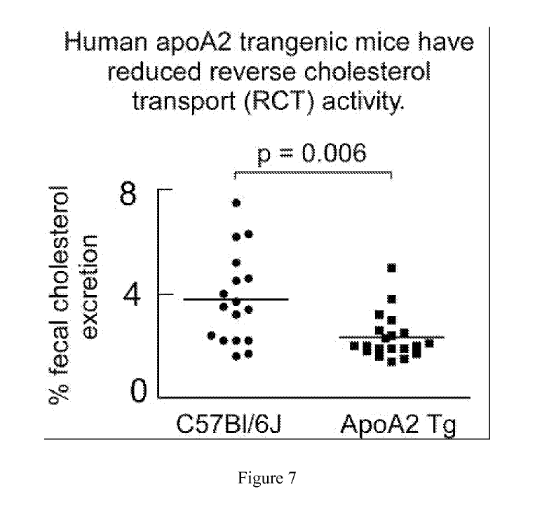

FIG. 7 shows ApoA2 transgenic mice show less reverse cholesterol transport compared to C57Bl/6J mice. C57Bl/6J or apoA2 transgenic mice were injected subcutaneously with cholesterol-loaded macrophages and the extent of radiolabel cholesterol and sitostenol (internal standard) tracers that appeared within feces were determined. Feces were collected for 48 h and percent fecal excretion (in vivo RCT) of labeled cholesterol was calculated as per Sehayek and hazen, ATVB, 2008). While apoA2 transgenic mice have higher plasma [14C] cholesterol compared to wild-type mice (not shown), they have reduced fecal excretion of cholesterol compared to wild-type controls, indicating overall impaired RCT.

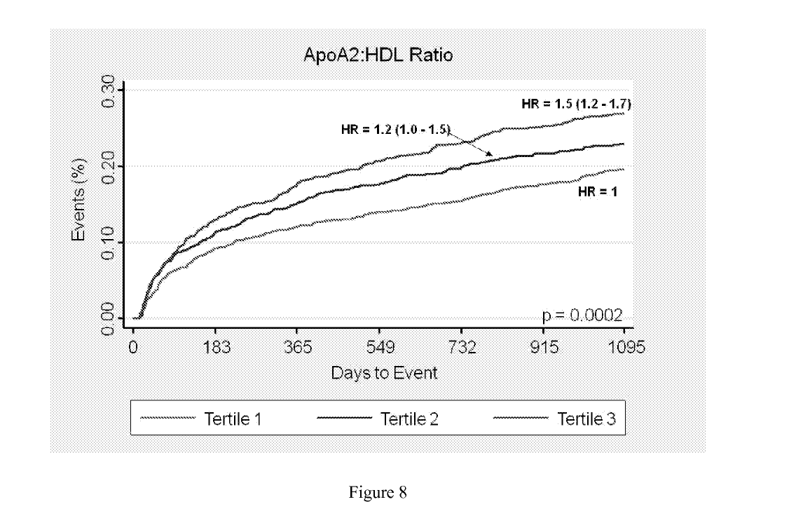

FIG. 8 shows that the apoA2/HDL cholesterol ratio—which is a measure of the apoA2 level per particle, is dose dependently associated with prospective risk of cardiovascular events (MI, stroke, death) over the ensuing 3 year period. Note that cumulative event rate continues to increase over time among the subjects with higher apoA2/HDLc level (third tertile is at 50% increase risk with hazard ratio of 1.5 and 95% CI of 1.2-1.7).

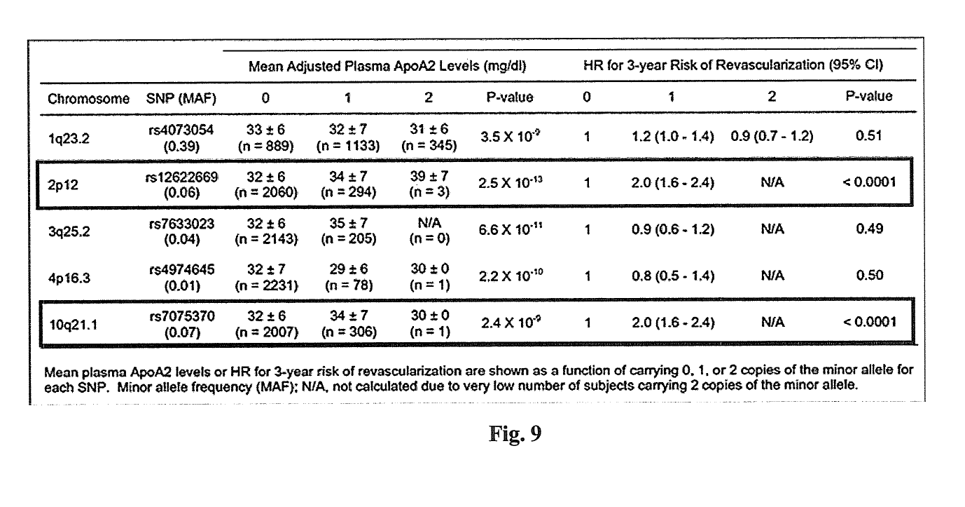

FIG. 9 shows that two polymorphisms that regulate apoA2 levels identified by GWAS (genome wide association studies) are strongly associated with future cardiovascular event risk (rs1262269 and rs70753770). In both cases, the at risk SNP (2.0 fold increased risk for future cardiovascular event defined as MI, stroke or death over ensuing 3 yr period) is associated with higher level of apoA2.

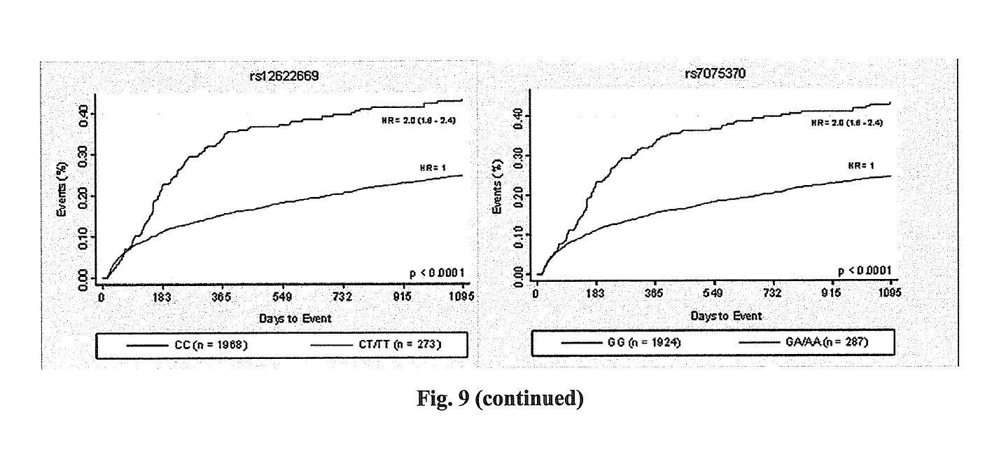

### DETAILED DESCRIPTION

The present inventions will now be described by reference to more detailed embodiments. These inventions may, however, be embodied in different forms and should not be construed as limited to the embodiments set forth herein. Rather these embodiments are provided so that this disclosure will be thorough and complete, and will convey the scope of the inventions to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

All publications, patent applications, patents, and other references mentioned herein are incorporated by reference in their entirety.

### ApoA2/HDLc As A Risk Marker For Cardiovascular Disease

ApoA2 is encoded by the ApoA2 gene. Identifying information for the apoA2 protein, gene, and mRNA are as follows: HGNC ID is HGNC:601; chromosome location is 1q21-q23; Entrez ID is 336; Ensembl ID is ENSG00000158874; UniProt ID is P02652; RefSeq (mRNA) ID is NM_{—}001643; and the RefSeq (protein) ID is NP_{—}001634. A mouse ortholog is also known (Mouse Genome Database ID MGI:88050), as is a rat ortholog (Rat Genome Database ID RGD:2131).

In some embodiments, the ratio of apoA2 to HDLc or equivalents thereof in a biological sample from a subject is compared to a control value that is derived from the ratio of apoA2 to HDLc or equivalents thereof in comparable biological samples obtained from a control population. In some embodiments an equivalent of HDLc may be used. For example, levels of HDLp, apoA1, or (apoA1+apoA2) can serve as equivalents for HDLc. It should be understood that values for HDLc can be determined using levels of HDLp, apoA1, or (apoA1+apoA2). In another embodiment, the present methods comprise determining the ratio of apoA2 mass to HDLc, HDLp, apoA1, or (apoA1+apoA2) in a biological sample, for example, blood, serum, or plasma, from the subject. In certain embodiments, the biological sample is blood, or a fluid derived from blood, e.g., serum, plasma, urine, or combinations thereof. Levels of HDLc, HDLp, apoA1, and apoA2 can be measured using methods known to those skilled in the art, and may include, but are not limited to, immunoanalysis, ELISA, and nuclear magnetic resonance (NMR).

In one embodiment, the comparison characterizes the subject's present risk of having CVD, as determined using standard protocols for diagnosing CVD. Moreover, the extent of the difference between the subject's ratio of apoA2 to HDLc or equivalents thereof and the control value is also useful for characterizing the extent of the risk and thereby determining which subjects would most greatly benefit from certain therapies. In another embodiment, the comparison characterizes the subject's risk of developing CVD in the future.

In another embodiment, the comparison can be used to characterize the subject's risk of experiencing a MACE, such as a need for revascularization, a myocardial infarction, stroke, heart failure, death, or combinations thereof, within the ensuing 3 months, 6, months, 1 year, 3 years, 5 years, or 10 years. The present methods can also be used to determine if a subject presenting with chest pain is at risk of experiencing a MACE, such as the need for revascularization, a myocardial infarction, reinfarction, death, or combinations thereof, near term, e.g., within the following 3 months, 6 months, 1 year after a subject presents with chest pain.

Also provided herein are methods for monitoring over time the status of CVD in a subject. In one embodiment, the method comprises determining the ratio of apoA2 to HDLc or equivalents thereof in a biological sample taken from a subject at an initial time and in a corresponding biological sample taken from a subject at a subsequent time. An increase in the ratio of apoA2 to HDLc or equivalents thereof in a biological sample taken at the subsequent time as compared to the initial time indicates that a subject's risk of having CVD has increased. A decrease in the ratio of apoA2 to HDLc or equivalents thereof indicates that the subject's risk of having CVD has decreased. For those subjects who have already experienced a MACE, such methods are also useful for assessing a subject's risk of experiencing a subsequent MACE. In such subjects, a decrease in levels of the ratio of apoA2 to HDLc or equivalents thereof indicates that the subject is at decreased risk of experiencing a subsequent MACE. An increase in the ratio of apoA2 to HDLc or equivalents thereof in a subject over time indicates that the subject's risk of experiencing a subsequent MACE has increased.

In another embodiment, the present disclosure provides a method for characterizing a subject's response to therapy directed at stabilizing or regressing CVD. The method comprises determining the ratio of apoA2 to HDLc or equivalents thereof in a biological sample taken from the subject prior to therapy (or therapeutic lifestyle change such as diet or exercise), and determining the ratio of apoA2 to HDLc or equivalents thereof in a corresponding biological sample taken from the subject during or following therapy or lifestyle change. A decrease in the ratio of apoA2 to HDLc or equivalents thereof in the sample taken after or during therapy or lifestyle change as compared to the ratio of apoA2 to HDLc or equivalents thereof in the sample taken before therapy is indicative of a positive effect of the therapy on cardiovascular disease in the treated subject.

In another embodiment, the present disclosure relates to kits that comprise reagents for assessing the ratio of apoA2 to HDLc or equivalents thereof in biological samples obtained from a test subject. In certain embodiments, the kits also comprise printed materials such as instructions for practicing the present methods, or information useful for assessing a test subject's risk of CVD. Examples of such information include, but are not limited cut-off values, sensitivities at particular cut-off values, as well as other printed material for characterizing risk based upon the outcome of the assay. In some embodiments, such kits may also comprise control reagents.

In certain embodiments, the ratio of apoA2 to HDLc or equivalents thereof in the bodily sample of the test subject is compared to a control value that is derived from the ratio of apoA2 to HDLc or equivalents thereof in comparable bodily samples of control subjects. Test subjects whose ratio of apoA2 to HDLc or equivalents thereof is above the control value or in the lower range of control values are at greater risk of having or developing CVD than test subjects whose ratio of apoA2 to HDLc or equivalents thereof is at or below the control value or in the higher range of control values. Moreover, the extent of the difference between the subject's ratio of apoA2 to HDLc or equivalents thereof and the control value is also useful for characterizing the extent of the risk and thereby, determining which subjects would most greatly benefit from certain therapies.

In certain embodiments, the subject's risk profile for CVD is determined by combining a first risk value, which is obtained by comparing the ratio of apoA2 to HDLc or equivalents thereof in a bodily sample of the subject with the ratio of apoA2 to HDLc or equivalents thereof in a control population, with one or more additional risk values to provide a final risk value. Such additional risk values may be obtained by procedures including, but not limited to, determining the subject's blood pressure, assessing the subject's response to a stress test, determining levels of myeloperoxidase, homocitrulline, C-reactive protein, low density lipoprotein, or cholesterol in a bodily sample from the subject, or assessing the subject's atherosclerotic plaque burden.

In one embodiment, the method is used to assess the test subject's risk of having cardiovascular disease. Medical procedures for determining whether a human subject has coronary artery disease or is at risk for experiencing a complication of coronary artery disease include, but are not limited to, coronary angiography, coronary intravascular ultrasound (IVUS), stress testing (with and without imaging), assessment of carotid intimal medial thickening, carotid ultrasound studies with or without implementation of techniques of virtual histology, coronary artery electron beam computer tomography (EBTC), cardiac computerized tomography (CT) scan, CT angiography, cardiac magnetic resonance imaging (MRI), and magnetic resonance angiography (MRA). Because cardiovascular disease, typically, is not limited to one region of a subject's vasculature, a subject who is diagnosed as having or being at risk of having coronary artery disease is also considered at risk of developing or having other forms of CVD such as cerebrovascular disease, aortic-iliac disease, heart failure, and peripheral artery disease. Subjects who are at risk of having cardiovascular disease are at risk of having an abnormal stress test or abnormal cardiac catheterization. Subjects who are at risk of having CVD are also at risk of exhibiting increased carotid intimal medial thickness and coronary calcification, characteristics that can be assessed using non-invasive imaging techniques. Subjects who are at risk of having CVD are also at risk of having an increased atherosclerotic plaque burden, a characteristic that can be examined using intravascular ultrasound.

In another embodiment, the present methods are used to assess the test subject's risk of developing CVD in the future. In one embodiment, the test subject is an apparently healthy individual. In another embodiment, the subject is not otherwise at elevated risk of having CVD.

In another embodiment, the present methods are used to assess the test subject's risk of experiencing a MACE within 3 months, 6 months, one year, three years, five years, or ten years. In another embodiment, the present methods are used to determine if a subject presenting with chest pain is at risk of experiencing the need for revascularization or another MACE, such as a myocardial infarction, reinfarction, heart failure, or death, near term after the subject presents with chest pain.

The present disclosure also provides a method for monitoring over time the status of CVD in a subject who has been diagnosed as having CVD. In this context, the method is also useful for monitoring the risk for atherosclerotic progression or regression in a subject with CVD. In one embodiment, the method comprises determining the ratio of apoA2 to HDLc or equivalents thereof in a biological sample taken from the subject at an initial time and in a corresponding biological sample taken from the subject at a subsequent time. An increase in the ratio of apoA2 to HDLc or equivalents thereof in a biological sample taken at the subsequent time as compared to the initial time indicates that the subject's risk for experiencing a MACE from the CVD has increased. A decrease in the ratio of apoA2 to HDLc or equivalents thereof indicates that the subject's risk for experiencing a MACE from the CVD has improved or regressed. For those subjects who have already experienced an acute adverse cardiovascular event such as a myocardial infarction or ischemic stroke, such method can also be used to assess the subject's risk of having a subsequent MACE. An increase over time in the ratio of apoA2 to HDLc or equivalents thereof in the subject indicates that a subject's risk of experiencing a subsequent adverse cardiovascular event has increased. A decrease over time in the ratio of apoA2 to HDLc or equivalents thereof in the subject indicates that that the subject's risk of experiencing a subsequent adverse cardiovascular event has decreased.

In another embodiment, the present disclosure provides a method for evaluating therapy in a subject suspected of having or diagnosed as having cardiovascular disease. The method comprises determining the ratio of apoA2 to HDLc or equivalents thereof in a biological sample taken from the subject prior to therapy and determining the ratio of apoA2 to HDLc or equivalents thereof in a corresponding biological sample taken from the subject during or following therapy. A decrease in the ratio of apoA2 to HDLc or equivalents thereof in the sample taken after or during therapy as compared to the ratio of apoA2 to HDLc or equivalents thereof in the sample taken before therapy is indicative of a positive effect of the therapy on cardiovascular disease in the treated subject.

### Biological Samples

Exemplary biological samples include, but are not necessarily limited to blood samples (e.g., blood, serum, plasma, and other blood-derived samples). The sample may be fresh blood or stored blood (e.g., in a blood bank) or blood fractions. The sample may be a blood sample expressly obtained for the assays of this invention or a blood sample obtained for another purpose which can be sub-sampled for the assays of this invention.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood such as heparin. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions, employing one of a variety of buffers, such as phosphate, Tris, or the like, at physiological pH can be used.

### Subjects

The subject is any human or other animal to be tested for characterizing its risk of CVD. In certain embodiments, the subject does not otherwise have an elevated risk of an adverse cardiovascular event. Subjects having an elevated risk of an adverse cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.).

In certain embodiments the subject is apparently healthy. “Apparently healthy,” as used herein, means individuals who have not previously been diagnosed as having any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. Apparently healthy individuals also do not otherwise exhibit symptoms of disease. In other words, such individuals, if examined by a medical professional, would be characterized as healthy and free of symptoms of disease.

In certain embodiments, the subject is a nonsmoker. “Nonsmoker” means an individual who, at the time of the evaluation, is not a smoker and has not had a tobacco product for the preceding 1 year period. This includes individuals who have never smoked as well as individuals who in the past have smoked but have not smoked for the past year. In certain embodiments, the subject is a smoker.

In some embodiments, the subject is a non-hyperlipidemic subject. A “non-hyperlipidemic” is a subject that is a non-hypercholesterolemic, a non-hypertriglyceridemic, or both. A “non-hypercholesterolemic” subject is one that does not fit the current criteria established for a hypercholesterolemic subject. A non-hypertriglyceridemic subject is one that does not fit the current criteria established for a hypertriglyceridemic subject (see, e.g., Harrison's Principles of Experimental Medicine, 15th Edition, McGraw-Hill, Inc., N.Y.). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL cholesterol (<40 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a non-hyperlipidemic subject is defined as one whose cholesterol and triglyceride levels are below the limits set as described above for both the hypercholesterolemic and hypertriglyceridemic subjects.

### Methods For Determining The Ratio Of ApoA2 To HDLc Or Equivalents Thereof

The level of apoA2 in the subject's blood, serum, or plasma can be determined using any method for determining levels of apoA2 in a subject's bodily fluid. In some embodiments, the level of apoA2 refers to apoA2 mass in the biological sample. ApoA2 mass levels in the biological sample may be determined using polyclonal or monoclonal antibodies that are immunoreactive with such protein. For example, antibodies immunospecific for apoA2 may be made and labeled using standard procedures and then employed in immunoassays to determine apoA2 in the sample. Suitable immunoassays include, by way of example, radioimmunoassays, both solid and liquid phase, fluorescence-linked assays, competitive immunoassays, and enzyme-linked immunosorbent assays. In certain embodiments, the immunoassays are also used to quantify the amount of apoA2 that is present in the sample.

Monoclonal antibodies raised against apoA2 may be produced according to established procedures. Generally, apoA2 is used to immunize a host animal. Suitable host animals, include, but are not limited to, rabbits, mice, rats, goats, and guinea pigs. Various adjuvants may be used to increase the immunological response in the host animal. The adjuvant used depends, at least in part, on the host species. Such animals produce heterogeneous populations of antibody molecules, which are referred to as polyclonal antibodies and which may be derived from the sera of the immunized animals.

Monoclonal antibodies, which are homogenous populations of an antibody that bind to a particular antigen, are obtained from continuous cells lines. Conventional techniques for producing monoclonal antibodies are the hybridoma technique of Köhler, G. & Milstein, C., Nature 1975 Aug. 7; 256(5517):495-7 and the human B-cell hybridoma technique of Kozbor, D. & Roder, J. C., Immunology Today 1983 March; 4(3):72-9. Such antibodies may be of any immunoglobulin class including IgG, IgM, IgE, IgA, IgD and any class thereof.

The term “high density lipoprotein” or “HDL” is defined in accordance with common usage of those of skill in the art. Generally “HDL” refers to a lipid-protein complex which when isolated by ultracentrifugation is found in the density range of d=1.063 to d=1.21. The concentration of HDL particles in a subject's blood, serum, or plasma can be determined by NMR. Alternatively, the concentration of HDL particles in a subject's blood, serum or plasma can be estimated by measuring the level of HDLc, HDLp, apoA1, or (apoA1+apoA2) in the subject's blood, serum, or plasma. ApoA1 is the major structural protein on HDL. It consists of a series of amphipathic helices that are functionally important for protein-lipid interactions as well as protein-protein interactions. The carboxy terminus of apoA1 has high lipid-binding capacity, while the amino terminus has limited lipid-binding capacity but may be important in protein-protein interaction (Frank, P. G. & Marcel, Y. L., J Lipid Res. 2000 June; 41(6):853-72). ApoA1 is largely responsible for mediating HDL assembly and is a determinant of HDL structure and composition. Levels of apoA1 and apoA2 in the subject's sample can be determined using polyclonal or monoclonal antibodies that are immunoreactive with such protein. For example, antibodies immunospecific for apoA1 may be made and labeled using standard procedures and then employed in immunoassays to determine apoA1 in the sample. Suitable immunoassays include, by way of example, radioimmunoassays, both solid and liquid phase, fluorescence-linked assays, competitive immunoassays, and enzyme-linked immunosorbent assays. In certain embodiments, the immunoassays are also used to quantify the amount of apoA1 that is present in the sample.

### Control Value

In certain embodiments, the ratio of apoA2 to HDLc or equivalents thereof in the biological sample obtained from the test subject may be compared to a control value. The control value is based upon the ratio of apoA2 to HDLc or equivalents thereof in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of an acute adverse cardiovascular event such as a myocardial infarction or stroke, evidence of atherosclerosis by diagnostic imaging methods including, but not limited to coronary angiography. In certain embodiments, the reference cohort is comprised of individuals, who if examined by a medical professional would be characterized as free of symptoms of disease. In another example, the reference cohort may be individuals who are nonsmokers. “Nonsmoker”, as used herein, means an individual who, at the time of the evaluation, is not a smoker and has not used a tobacco product for the preceding 1 year period. This includes individuals who have never smoked as well as individuals who in the past have smoked but has not smoked for the past year. A nonsmoker cohort may have a different normal ratio of apoA2 to HDLc or equivalents thereof than will a smoking population or the general population. Accordingly, the control values selected may take into account the category into which the test subject falls. Appropriate categories can be selected with no more than routine experimentation by those of ordinary skill in the art. As further information becomes available as a result of routine performance of the methods described herein, population average values for the ratio of apoA2 to HDLc or equivalents thereof may be used. In other embodiments, “normal” ratios of apoA2 to HDLc or equivalents thereof may be obtained by determining the ratio of apoA2 to HDLc or equivalents thereof in samples obtained from subjects without CVD, subjects who do not develop CVD in a prescribed period of time, from archived patient samples, and the like.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Control values of the ratio of apoA2 to HDLc or equivalents thereof in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G. & Miller, M. C., Clinical epidemiology and biostatistics, Malvern, Pa.: Williams & Wilkins; Harwal Pub. Co.; 1992 (ISBN 0683062069), which is specifically incorporated herein by reference. A “cutoff” value can be determined for each risk marker that is assayed.

### Comparison Of The Ratio Of Apo2 To HDLc Or Equivalents Thereof From The Test Subject To The Control Value

The ratio of apoA2 to HDLc or equivalents thereof in the individual's biological sample may be compared to a single control value or to a range of control values. If the level of the present risk marker in the test subject's biological sample is greater than the control value or exceeds or is in the upper range of control values, the test subject is at higher risk of developing or having CVD than individuals with levels below the control value or in the lower range of control values. In contrast, if the ratio of apoA2 to HDLc or equivalents thereof in the test subject's biological sample is below the control value or is in the lower range of control values, the test subject is at lower risk of developing or having CVD than individuals whose levels are comparable to or above the control value or in the upper range of control values. The extent of the difference between the test subject's risk marker levels and control value is also useful for characterizing the extent of the risk and thereby, determining which individuals would most greatly benefit from certain aggressive therapies. In those cases, where the control value ranges are divided into a plurality of groups, such as the control value ranges for individuals at high risk, average risk, and low risk, the comparison involves determining into which group the test subject's level of the relevant risk marker falls.

The present predictive tests are useful for determining if and when therapeutic agents that are targeted at preventing CVD or for slowing the progression of CVD should and should not be prescribed for an individual. For example, individuals with ratios of apoA2 to HDLc or equivalents thereof below a certain cutoff value, or that are in the lower tertile or quartile of a “normal range,” could be identified as those in need of less aggressive or even no intervention with lipid lowering agents, life style changes, or combinations thereof.

### Evaluation Of CVD Therapeutic Agents

Also provided are methods for evaluating the effect of CVD therapeutic agents on individuals who have been diagnosed as having or as being at risk of developing CVD. Such therapeutic agents include, but are not limited to, anti-inflammatory agents, insulin sensitizing agents, antihypertensive agents, anti-thrombotic agents, anti-platelet agents, fibrinolytic agents, lipid reducing agents, direct thrombin inhibitors, CDTP inhibitor thioglytizone, glycoprotein II b/IIIa receptor inhibitors, agents directed at raising or altering HDL metabolism such as apoA1 milano or CETP inhibitors, or agents designed to act as artificial HDL. Such evaluation comprises determining the ratio of apoA2 to HDLc or equivalents thereof in a biological sample taken from the subject prior to administration of the therapeutic agent and a corresponding biological fluid taken from the subject following administration of the therapeutic agent. A decrease in the level of the selected risk markers in the sample taken after administration of the therapeutic as compared to the level of the selected risk markers in the sample taken before administration of the therapeutic agent is indicative of a positive effect of the therapeutic agent on cardiovascular disease in the treated subject.

### Kits

Also provided are kits for practicing the present methods. Such kits contain reagents for assessing levels of apoA2 and HDLc or equivalents thereof in a biological sample. In one embodiment, the kit comprises a reagent, e.g., an antibody, for measuring apoA2 and a reagent, e.g., an antibody, for measuring apoA1 levels in the subject's bodily sample. In one embodiment, the kit also comprises instructions for using the reagent in the present methods. In another embodiment, the kit comprises information useful for determining a subject's risk of cardiovascular disease or a complication. Examples of such information include, but are not limited cut-off values, sensitivities at particular cut-off values, as well as other printed material for characterizing risk based upon the outcome of the assay.

### EXAMPLES

Methods: We examined the relationship between plasma ApoA2 (adjusted for HDL cholesterol) with prevalent cardiovascular diseases (CVD) as well as need for future revascularization up to 3-year follow-up in 4,670 subjects undergoing elective coronary angiography without acute coronary syndromes.

Results: In our cohort (age 63±11 years, 66% male, 79% with CVD, 32% diabetes mellitus), mean ApoA2 level was 32±7 mg/dL and highly correlated with HDL (Spearman's r=0.55, p<0.001). While unadjusted ApoA2 levels were inversely associated with greater need for revascularization at 3 years (Hazard ratio [HR] 0.91, 95% confidence interval [CI] 0.87-0.96, p<0.001), higher ApoA2/HDL ratio (mean 0.97±0.23) portends higher rather than lower prevalence of CVD (odds ratio [OR] 1.54; 95% CI 1.42-1.66, p<0.001) and need for future revascularization (HR 1.18; 95% CI 1.13-1.24, p<0.001). After adjusting for traditional risk factors, ApoA2/HDL remained independently predictive of prevalent CVD (OR 1.47, 95% CI 1.35-1.61, p<0.001) and need for future revascularization (HR 1.15, 95% CI 1.09-1.21, p<0.001).

Conclusion: In stable cardiac patients, ApoA2 (when adjusted for HDL) provides independent association with CVD and risk prediction of need for future revascularization, indicating a direct association between ApoA2 with atherosclerotic burden and progression rather than with cardioprotection in humans.

1. A method for characterizing a subject's risk of having cardiovascular disease, comprising:
determining the ratio of apoA2 to HDLc or equivalents thereof in a biological sample from the subject,
comparing the ratio of apoA2 to HDLc or equivalents thereof in a biological sample from the subject to a control value or an internal standard,
wherein the biological sample is blood, serum, or plasma
wherein a subject whose ratio of apoA2 to HDLc or equivalents thereof is high compared to a control value or an internal standard indicates that the subject is at risk of having cardiovascular disease.

2. The method of claim 1, wherein the HDLc equivalent is HDLp.

3. The method of claim 1, wherein the HDLc equivalent is apoA1.

4. The method of claim 1, wherein the HDLc equivalent is obtained by determining both apoA1 and apoA2 levels in the sample.

5. The method of claim 2, wherein a value for HDLp is determined by NMR.

6. The method of claim 1, wherein a value for apoA2 is determined by use of an antibody.

7. The method of claim 1, wherein levels of apoA2 are compared to a control value or a range of control values based upon levels of apoA2 in comparable biological samples from a control population of human subjects.

8. (canceled)

9. (canceled)

10. A method of characterizing the risk of experiencing a subsequent acute cardiovascular event in a subject who has experienced one or more acute adverse cardiovascular events, comprising:
determining the ratio of apoA2 to HDLc or equivalents thereof in a biological sample taken from the subject at an initial time and in a corresponding biological sample taken from the subject at a subsequent time,
comparing the ratio of apoA2 to HDLc or equivalents thereof in a biological sample taken from the subject at an initial time to the ratio of apoA2 to HDLc or equivalents thereof in a biological sample taken from the subject at a subsequent time,
wherein an increase in the ratio of apoA2 to HDLc or equivalents thereof in a biological sample taken at the subsequent time as compared to the initial time indicates that a subject's risk of experiencing a subsequent adverse cardiovascular event has increased.

11. (canceled)

12. A method for characterizing a subject's risk of having cardiovascular disease, comprising:
identifying at least one genetic determinant of apoA2 level in a test subject, the genetic determinant selected from the group consisting of rs1262269 and rs70753770 SNPs,
wherein, if the test subject carries even one allele of an at risk SNP, which is associated with higher apoA2 levels, the test subject is at increased risk of having or developing CVD, and therefore at increased risk of experiencing CVD complications, a MACE, or both.

13. The method of claim 1, wherein a subject whose ratio of apoA2 to HDLc or equivalents thereof is high compared to a control value or an internal standard indicates that the subject is at risk of developing cardiovascular disease or experiencing a major adverse cardiac event within 3 years.

14. The method of claim 1, wherein the ratio of apoA2 to HDLc for the subject comprises a first risk value; and
determining one or more additional cardiovascular risk values in the subject, wherein said one or more additional risk values are obtained by at least one of:
a) determining the subject's blood pressure;
b) determining levels of low density lipoprotein, or cholesterol, or both in a biological sample from the subject;
c) assessing the subject's response to a stress test;
d) determining levels of myeloperoxidase, C-reactive protein, or both in a biological sample from the subject; and
e) determining the subject's atherosclerotic plaque burden, and
combining said first risk value with said one or more additional risk values to provide a final risk value.
